(19) United States
(12) Patent Application Publication
(10) Pub. No.: US 2009/0197919 A1
(43) Pub. Date: Aug. 6, 2009

(54) Novel Method for Preparation of Ammonium Salts of Esomeprazole

(76) Inventors: Rolf Bergman, Molndal (SE); Christina Fregler, Molndal (SE); Per Lindberg, Molndal (SE)

Correspondence Address: WHITE & CASE LLP;PATENT DEPARTMENT, 1155 AVENUE OF THE AMERICAS, NEW YORK, NY, 10036, US

(21) Appl. No.: 12/303,332

(22) PCT Filed: Jun. 7, 2007
(86) PCT No.: PCT/SE2007/000548

§ 371 Date: Dec. 3, 2008

Related U.S. Application Data
(60) Provisional application No. 60/811,698, filed on Jun. 7, 2006.

(51) Int. Cl. A61K 31/4439; C07D 401/12

(52) U.S. Cl. 514/338; 546/273.4

(57) ABSTRACT

The present invention relates to a process for the preparation of quartenary ammoniumsalts of esomeprazole. Further, the present invention also relates to the use quartenary ammoniumsalts of esomeprazole for the treatment of gastrointestinal disorders, pharmaceutical compositions containing them as well as the quartenary ammoniumsalts of esomeprazole, as such.

### FIELD OF THE INVENTION

The present invention relates to a process for synthesis of salts of (S)-5-methoxy-2-[[(4-methoxy-3,5-dimethyl-2-pyridinyl)-methyl]sulfinyl]-1H-benzimidazole (esomeprazole), in a pure and isolated form.

### BACKGROUND OF THE INVENTION AND PRIOR ART

The compound 5-methoxy-2-[[(4-methoxy-3,5-dimethyl-2-pyridinyl)methyl]sulfinyl]-1H-benzimidazole, having the generic name omeprazole, and therapeutically acceptable salts thereof, are described in EP 0 005 129.

Omeprazole is a sulfoxide and a chiral compound, wherein the sulphur atom being the stereogenic center. Thus, omeprazole is a racemic mixture of its two single enantiomers, the R- and S-enantiomer of omeprazole, herein referred to as R-omeprazole and S-omeprazole, the latter have the generic name esomeprazole. The absolute configuration of the enantiomers of omeprazole has been determined by an X-ray study of an N-alkylated derivate of the R-enantiomer.

Omeprazole and esomeprazole are proton pump inhibitors, and are useful as antiulcer agents. In a more general sense, omeprazole and esomeprazole may be used for prevention and treatment of gastric acid related diseases in mammals and especially in man. Specific alkaline salts of omeprazole are disclosed in EP 0 124 495. Herein, quaternary ammonium salts and guanidine salts of omeprazole are disclosed. Document WO 97/41114 discloses processes for preparing magnesium salt of benzimidazoles, including magnesium salt of omeprazole.

Certain salts of the single enantiomers of omeprazole and their preparation are disclosed in WO 94/27988, for instance, quaternary ammonium salts of esomeprazole are mentioned. The described salts of esomeprazole have improved pharmacokinetic and metabolic properties, which will give an improved therapeutic profile such as a lower degree of interindividual variation. WO 96/02535 and WO 98/54171 disclose preferred processes for preparing esomeprazole and salts thereof.

In the formulation of drug compositions, it is important for the active pharmaceutical ingredient to be in a form in which it can be conveniently handled and processed. This is of importance, not only from the point of view of obtaining a commercially viable manufacturing process, but also from the point of view of subsequent manufacture of pharmaceutical formulations (e.g. oral dosage forms such as tablets) comprising the active pharmaceutical ingredient.

Further, in the manufacture of oral pharmaceutical compositions, it is important that a reliable, reproducible and constant plasma concentration profile of the active pharmaceutical ingredient is provided following administration to a patient.

Chemical stability, solid state stability, and “shelf life” of the active pharmaceutical ingredient are important properties for a pharmaceutical active compound. The active pharmaceutical ingredient, and compositions containing it, should be capable of being effectively stored over appreciable periods of time, without exhibiting a significant change in the physico-chemical characteristics of the active pharmaceutical ingredient, e.g. its chemical composition, density, hygroscopicity and solubility.

### DESCRIPTION OF THE INVENTION

The present invention refers to a process for preparing a quartenary ammonium salt of esomeprazole of formula I

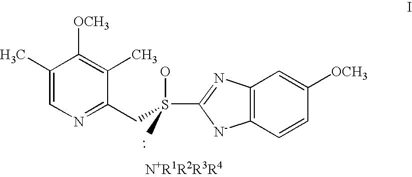

wherein
R₁, R₂, R₃ and R₄ are individually selected from
(A) C₁-C₁₄ alkyl group, which alkyl group is optionally substituted by one or more groups selected from amino, hydroxy, halogen, R₅O—, C₃-C₁₂ cycloalkyl (which cycloalkyl is optionally substituted by one or more groups selected from C₁-C₃ alkyl, hydroxy, C₁-C₃ alkoxy, halogen, oxo, R₂₃ₐOC(O)—, (R_{23b})(R_{23c})NC(O)—, R_{23d}C(O)N(R₂₃ₑ)—, R_{23f}C(O)O—, R_{23g}OC(O)—NH—, (R₂₃ₕ)(R₂₃ⱼ)NC(O)O—), aryl or Het¹ (both groups optionally substituted by one to three groups selected from C₁-C₇ alkyl, hydroxy, —CH₂OH, halogen, oxo, nitro, C₁-C₇ alkoxy, R₂₄ₐOC(O)—, (R_{24b})(R_{24c})NC(O)—, R_{24d}C(O)N(R₂₄ₑ)—, R_{24f}C(O)O—, R_{24g}OC(O)—NH—, (R₂₄ₕ)(R₂₄ⱼ)NC(O)O—, aryl, Het or R₂₅C(O)— (which aryl and Het are optionally substituted by one or two halogens, C₁-C₄ alkyl, hydroxy C₁-C₄alkyl, C₁-C₄ alkoxy, hydroxy C₁-C₄ alkoxy, nitro); R₆—O—(CH₂)ₘ—O—, R₇ₐOC(O)—, (R_{7b})(R_{7c})NC(O)—, R_{7d}C(O)N(R₇ₑ)—, R_{7f}C(O)O—, R_{7g}C(O)S—, R₇ₕOC(O)N(R₇ⱼ)—, (R₇ₖ)(R₇₁)NC(O)O—, R₇ₘOC(O)O—, R₈—SO₂—NH—, phtalimido, succinimido, R₉C(O)—, R₁₀—(CH₂)ₙ—C(O)— or (R₁₁ₐ)(R_{11b})(R_{11c})C—C(O)O—;
(B) aryl or Het (both groups optionally substituted by one to three groups selected from C₁-C₇ alkyl, hydroxy, C₁-C₇ alkoxy, halogen, R₁₂ₐOC(O)—, (R_{12b})(R_{12c})NC(O)—, R_{12d}C(O)N(R₁₂ₑ)—, R_{12f}C(O)O—, R_{12g}OC(O)NR₁₂ₕ—, (R₁₂ⱼ)(R₁₂ₖ)NC(O)O—, aryl, benzoyl or Het⁴), R₁₃C(O)— or (R₁₄ₐ)(R_{14b})N—); or R₁ and R₂ together may represent a cyclic structure containing 5-14 members, optionally substituted by one or more groups selected from hydroxy, oxo, C₁-C₇ alkyl (which alkyl group is optionally substituted by one or more groups selected from hydroxy, halogen, aryl or Het⁷), R₁₅O—, R₁₆ₐOC(O)—, (R_{16b})(R_{16c})NC(O)—, R_{16d}C(O)N(R₁₆ₑ)—, R_{16f}C(O)O—, R_{16g}OC(O)NR₁₆ₕ—, (R₁₆ⱼ)(R₁₆)NC(O)O—, R₁₇C(O)—, aryl or Het (which aryl or Het⁵ are optionally substituted by one or more of C₁-C₇ alkyl, hydroxy, oxo, C₁-C₇ alkoxy, halogen, R₂₆ₐOC(O)—, (R_{26b})(R_{26c})NC(O)—, R_{26d}C(O)N(R₂₆ₑ)—, R_{26f}C(O)O—, R_{26g}OC(O)NH—, (R₂₆ₕ)(R₂₆ⱼ)NC(O)O—, phenyl or benzoyl (which phenyl or benzoyl are optionally substituted by one or two halogens or C₁-C₅ alkyl C(O)O—)), phtalimido, succinimido or (R₁₈ₐ)(R_{18b})(R_{18c})C—C(O)O—;
or R₁, R₂ and R₃ together may represent a cyclic structure containing 5-16 members, optionally substituted by one or more groups selected from hydroxy, oxo, C₁-C₇ alkyl (which alkyl group is optionally substituted by one or more groups selected from hydroxy, halogen, oxo, aryl or Het⁸), R₁₉O—, R₂OC(O)—, aryl or Het (which aryl or Het are optionally substituted by one to three groups selected from C₁-C₇ alkyl, hydroxy, C₁-C₇ alkoxy, halogen, oxo, R₂₇ₐOC(O)—, (R_{27b})(R_{27c})NC(O)—, R_{27d}C(O)N(R₂₇ₑ)—, R_{27f}C(O)O—, R_{27g}OC(O)—NH—, (R₂₇ₕ)(R₂₇ⱼ)NC(O)O—, phenyl or benzoyl), R₂₁ₐOC(O)—, (R_{21b})(R_{21c})NC(O)—, R_{21d}C(O)N(R₂₁ₑ)—, R_{21f}C(O)O—, R_{21g}OC(O)—NR₂₁ₕ—, (R₂₁ⱼ)(R₂₁ₖ)NC(O)O—, phtalimido, succinimido or (R₂₂ₐ)(R_{22b})(R_{22c}) C—C(O)O—;
wherein
R₅ is selected from C₁-C₆ alkyl, aryl, Het⁹ (which groups are optionally substituted by one or more groups selected from hydroxy, halogen, C₁-C₆ alkoxy);
R₆ is selected from aryl or Het (both groups optionally substituted by one or more groups selected from C₁-C₈ alkyl, hydroxy, C₁-C₇ alkoxy, halogen, R₂₈ₐOC(O)—, (R_{28b})(R_{28c})NC(O)—, R_{28d}C(O)N(R₂₈ₑ)—, R_{28f}C(O)O—, R_{28g}OC(O)—NH—, (R₂₈ₕ)(R₂₈ⱼ)NC(O)O—, aryl, benzoyl or Het¹¹);
R₇ₐ to R₇ₘ are independently selected, at each occurrence, from hydrogen, C₁-C₇ alkyl, aryl or Het¹² (which C₁-C₇ alkyl, aryl and Het¹² are optionally substituted by one or more groups selected from C₁-C₆ alkyl, hydroxy, C₁-C₃ alkoxy, halogen, R₂₉ₐOC(O)—, (R_{29b})(R_{29c})NC(O)—, R_{29d}C(O)N(R₂₉ₑ)—, R_{29f}C(O)O—, R_{29g}OC(O)—NH—, (R₂₉ₕ)(R₂₉ⱼ)NC(O)O—, aryl, benzoyl or Het¹³);
R₈ is selected from C₁-C₆ alkyl, aryl or Het (which groups are optionally substituted by one or more groups selected from C₁-C₆ alkyl);
R₉ is selected from linear or branched C₁-C₁₂ alkyl (optionally substituted by R₃₀OC(O)—), C₃-C₁₂ cycloalkyl (which cycloalkyl group is optionally further substituted by one or more groups selected from C₁-C₃ alkyl, hydroxy, C₁-C₃ alkoxy, halogen, R₃₁ₐOC(O)—, (R_{31b})(R_{31c})NC(O)—, R_{31d}C(O)NR₃₁ₑ—, R_{31f}C(O)O—, R_{31g} C(O)N(R₃₁ₕ)—, (R₃₁ⱼ)(R₃₁ₖ)NC(O)O—), aryl, benzoyl or Het¹⁵), aryl or Het¹⁶ (which aryl and Het¹⁶ are optionally substituted by one to three of the groups selected from C₁-C₆ alkyl, hydroxy, C₁-C₃ alkoxy, ethylenedioxy, halogen, R₃₂ₐOC(O)—, (R_{32b})(R_{32c})NC(O)—, R_{32d}C(O)NR₃₂ₑ—, R_{32f}C(O)O—, R_{32g}OC(O)NH—, (R₃₂ₕ)(R₃₂ⱼ)NC(O)O—), aryl, benzoyl or Het¹⁷);
R₁₀ is selected from aryl and Het¹⁸ (which groups are optionally substituted by one to three groups selected from C₁-C₃ alkyl, hydroxy, C₁-C₃ alkoxy, halogen, —COOH, ethylenedioxy);

R₁₁ₐ is selected from hydroxy or —CH₂OH;

R_{11b} is phenyl (optionally substituted by one to three groups selected from C₁-C₃ alkyl, hydroxy, C₁-C₃ alkoxy, halogen, R₃₃ₐOC(O)—, (R_{33b})(R_{33c})NC(O)—, R_{33d}C(O)N(R₃₃ₑ)—, R_{33f}C(O)O—, R_{33g}OC(O)—NH—, (R₃₃ₕ)(R₃₃ⱼ)NC(O)O—;

R_{11c} is selected from hydrogen, C₅-C₆ cycloalkyl or phenyl (which groups are optionally substituted by one to three groups selected from C₁-C₃alkyl, hydroxy, C₁-C₃alkoxy, halogen, R₃₄ₐOC(O)—, (R_{34b})(R_{34c})NC(O)—, R_{34d}C(O)N(R₃₄ₑ)—, R_{34f}C(O)O—, R_{34g}OC(O)NH—, (R₃₄ₕ)(R₃₄ⱼ)NC(O)O—);
R₁₂ₐ to R₁₂ₖ are independently selected, at each occurrence, from hydrogen, C₁-C₇alkyl, aryl, Het 9 (which groups are optionally substituted by one or more groups selected from C₁-C₆alkyl, hydroxy, C₁-C₃alkoxy, halogen, R₃₅ₐOC(O)—, (R_{35b})(R_{35c})NC(O)—, R_{35d}C(O)N(R₃₅ₑ)—, R_{35f}C(O)O—, R_{35g}OC(O)—NH—, (R₃₅ₕ)(R₃₅ⱼ)NC(O)O—, aryl, benzoyl or Het²⁰);
R₁₃ is selected from hydrogen or C₁-C₆ alkyl;
R₁₄ₐ to R_{14b} are independently selected, at each occurrence, from hydrogen or C₁-C₆ alkyl;
R₁₅ is selected from C₁-C₆ alkyl, aryl or Het²¹ (which groups are optionally substituted by one or more groups selected from hydroxy, halogen or C₁-C₆ alkoxy);
R₁₆ₐ to R₁₆ₖ are independently selected from, at each occurrence, hydrogen, C₁-C₇ alkyl, aryl or Het²² (which groups are optionally substituted by one or more groups selected from C₁-C₆ alkyl, hydroxy, C₁-C₃ alkoxy, halogen, R₃₆ₐOC(O)—, (R_{36b})(R_{36c})NC(O)—, R_{36d}C(O)N(R₃₆ₑ)—, R_{36f}C(O)O—, R_{36g}OC(O)—NH—, (R₃₆ₕ)(R₃₆ⱼ)NC(O)O—, aryl, benzoyl or Het²³);
R₁₇ is selected from hydrogen or C₁-C₆ alkyl;
R₁₈ₐ is selected from hydroxy or —CH₂OH;
R_{18b} is phenyl (optionally substituted by one to three groups selected from C₁-C₃ alkyl, hydroxy, C₁-C₃ alkoxy, halogen, R₃₇ₐOC(O)—, (R_{37b})(R_{37c})NC(O)—, R_{37d}C(O)N(R₃₇ₑ)—, R_{37f}C(O)O—, R_{37g}OC(O)—NH—, (R₃₇ₕ)(R₃₇ⱼ)NC(O)O—);
R_{18c} is selected from hydrogen, C₅-C₆ cycloalkyl or phenyl (which groups are optionally substituted by one to three groups selected from C₁-C₃ alkyl, hydroxy, C₁-C₃ alkoxy, halogen, R₃₈ₐOC(O)—, (R_{38b})(R_{38c})NC(O)—, R_{38d}C(O)N(R₃₈ₑ)—, R_{38f}C(O)O—, R_{38g}OC(O)—NH—, (R₃₈ₕ(R₃₈ⱼ)NC(O)O—;
R₁₉ is selected from C₁-C₆ alkyl, aryl or Het²⁴ (which groups are optionally substituted by one or more groups selected from hydroxy, halogen, C₁-C₆ alkoxy);
R₂₀ is selected from hydrogen and C₁-C₆ alkyl;
R₂₁ₐ to R₂₁ₖ are independently selected, at each occurrence, from hydrogen, C₁-C₇ alkyl, aryl or Het²⁵ (which groups are optionally substituted by one or more groups selected from C₁-C₆ alkyl, hydroxy, C₁-C₃ alkoxy, halogen, R₃₉ₐOC(O)—, (R_{39b})(R_{39c})NC(O)—, R_{39d}C(O)N(R₃₉ₑ)—, R_{39f}C(O)O—, R_{39g}OC(O)—NH—, (R₃₉ₕ)(R₃₉ⱼ)NC(O)O—, aryl, benzoyl or Het²⁶);
R₂₂ₐ is selected from hydroxy or —CH₂OH;
R_{22b} is phenyl (optionally substituted by one to three groups selected from C₁-C₃ alkyl, hydroxy, C₁-C₃ alkoxy, halogen, R₄₀ₐOC(O)—, (R_{40b})(R_{40c})NC(O)—, R_{40d}C(O)N(R₄₀ₑ)—, R_{40f}C(O)O—, R_{40g}OC(O)NH—, (R₄₀ₕ)(R₄₀ⱼ)NC(O)O—);
R_{22c} is selected from hydrogen, C₅-C₆ cycloalkyl or phenyl (which optionally is substituted by one to three groups selected from C₁-C₃ alkyl, hydroxy, C₁-C₃ alkoxy, halogen, R₄₁ₐOC(O), (R_{41b})(R_{41c})NC(O)—, R_{41d}C(O)N(R₄₁ₑ)—, R_{41f} C(O)O—, R_{41g}OC(O)NH—, (R₄₁ₕ)(R₄₁ⱼ)NC(O)O—);
R₂₃ₐ to R₂₃ⱼ are independently selected, at each occurrence, from hydrogen or C₁-C₆alkyl;
R₂₄ₐ to R₂₄ⱼ are independently selected, at each occurrence, from hydrogen or C₁-C₆alkyl;
R₂₅ is selected from C₁-C₄alkyl, aryl or Het²⁷ (which aryl and Het²⁷ are optionally substituted by one or two halogens, C₁-C₄ alkyl, hydroxy C₁-C₄alkyl, C₁-C₄ alkoxy, hydroxy C₁-C₄ alkoxy, nitro);
R₂₆ₐ to R₂₆ⱼ are independently selected, at each occurrence, from hydrogen or C₁-C₆alkyl;
R₂₇ₐ to R₂₇ⱼ are independently selected, at each occurrence, from hydrogen or C₁-C₆alkyl;
R₂₈ₐ to R₂₈ⱼ are independently selected, at each occurrence, from hydrogen or C₁-C₆alkyl;
R₂₉ₐ to R₂₉ⱼ are independently selected, at each occurrence, from hydrogen or C₁-C₆alkyl;
R₃₀ is selected from hydrogen or C₁-C₆ alkyl;
R₃₁ₐ to R₃₁ₖ are independently selected, at each occurrence, from hydrogen or C₁-C₆alkyl;
R₃₂ₐ to R_{32c} are independently selected, at each occurrence, from hydrogen or C₁-C₆alkyl;
R₃₃ₐ to R₃₃ⱼ are independently selected, at each occurrence, from hydrogen or C₁-C₆alkyl;
R₃₄ₐ to R₃₄ⱼ are independently selected, at each occurrence, from hydrogen or C₁-C₆alkyl;
R₃₅ₐ to R₃₅ⱼ are independently selected, at each occurrence, from hydrogen or C₁-C₆alkyl;
R₃₆ₐ to R₃₆ⱼ are independently selected, at each occurrence, from hydrogen or C₁-C₆alkyl;
R₃₇ₐ to R₃₇ⱼ are independently selected, at each occurrence, from hydrogen or C₁-C₆alkyl;
R₃₈ₐ to R₃₈ⱼ are independently selected, at each occurrence, from hydrogen or C₁-C₆alkyl;
R₃₉ₐ to R₃₉ⱼ are independently selected, at each occurrence, from hydrogen or C₁-C₆alkyl;
R₄₀ₐ to R₄₀ⱼ are independently selected, at each occurrence, from hydrogen or C₁-C₆alkyl;
R₄₁ₐ to R₄₁ⱼ are independently selected, at each occurrence, from hydrogen or C₁-C₆alkyl;
m is an integer selected from 1 to 5;
n is an integer selected from 1 to 3.

The compound of formula I as defined by formula I is hereby defined as the compound of the invention.

The process for preparing the quaternary ammoniumsalts esomeprazole of formula I comprises the following steps:

(i): mixing esomeprazole and N⁺(R₁)(R₂)(R₃)(R₄) X⁻;
wherein R₁, R₂, R₃ and R₄ are as defined above; X⁻ is selected from Cl⁻, Br⁻, I⁻, carboxylates, sulphonates, HSO₄⁻ and OH⁻; in an aqueous solvent system substantially saturated with potassium carbonate;
(ii): adding a water immiscible chlorinated hydrocarbon solvent;
(iii): isolating the organic phase;
(iv): recovering of the compound of formula I.

In one embodiment of the invention the reaction of the esomeprazole and the N⁺(R₁)(R₂)(R₃)(R₄) X⁻ as defined above is performed in an aqueous solvent substantially saturated with potassium carbonate (K₂CO₃).

By “substantially saturated” it is meant a solution comprising equal or more than 40% by weight potassium carbonate in an aqueous solvent, for example more than 45, 50 or 55% by weight.

In one embodiment of the invention, the aqueous solvent system in step (i) is saturated with potassium carbonate, i.e. comprises about 56% by weight potassium carbonate.

In one embodiment of the invention the esomeprazole and the quartenary ammoniumsalt of formula N⁺(R₁)(R₂)(R₃)(R₄) X⁻ are in step (i) added in equimolar amounts.

The aqueous solvent system may be selected from water or water soluble solvents, such as alcohols, ethers, amides, nitrites soluble in water; or mixtures thereof. Examples of water soluble solvents are methanol, ethanol, dioxane, tetrahydrofuran, acetonitril and DMF.

In one embodiment the aqueous solvent system is water.

The water immiscible solvent forming the organic phase are selected from solvents such as chlorinated solvents suitable for phase transfer. The solvent must also be stable in the presence of base, i.e. for the present invention the solvent should not degrade more than to some extent in the presence of the potassium carbonate. Examples of chlorinated solvents are dichloromethane, trichloromethane and 1,2-dichloroethane.

In one embodiment the compound of the invention is a quartenary alkyl ammoniumsalt of esomeprazole of formula I wherein R₁ is selected from

(A) C₁-C₁₄ alkyl group, which alkyl group is optionally substituted by one or more groups selected from amino, hydroxy, halogen, R₅O—, C₃-C₁₂ cycloalkyl (which cycloalkyl is optionally substituted by one or more groups selected from C₁-C₃ alkyl, hydroxy, C₁-C₃ alkoxy, halogen, oxo, R₂₃ₐOC(O)—, (R_{23b})(R_{23c})NC(O)—, R_{23d}C(O)N(R₂₃ₑ)—, R_{23f}C(O)O—, R_{23g}OC(O)—NH— or (R₂₃ₕ)(R₂₃ⱼ)NC(O)O—), aryl or Het (both groups optionally substituted by one to three groups selected from C₁-C₇ alkyl, hydroxy, —CH₂OH, halogen, oxo, nitro, C₁-C₇ alkoxy, R₂₄ₐOC(O)—, (R_{24b})(R_{24c})NC(O)—, R_{24d}C(O)N(R₂₄ₑ)—, R_{24f}C(O)O—, R_{24g}OC(O)—NH—, (R₂₄ₕ)(R₂₄ⱼ)NC(O)O—, aryl, Het³ or R₂₅C(O)— (which aryl and Het³ are optionally substituted by one or two halogens, C₁-C₄ alkyl, hydroxy C₁-C₄alkyl, C₁-C₄ alkoxy, hydroxy C₁-C₄ alkoxy, nitro); R₆—O—(CH₂)ₘ—O—, R₇ₐOC(O)—, (R_{7b})(R_{7c})NC(O)—, R_{7d}C(O)N(R₇ₑ)—, R_{7f}C(O)O—, R_{7g}C(O)S—, R₇ₕOC(O)N(R₇ⱼ)—, (R₇ₖ)(R₇₁)NC(O)O—, R₇ₘOC(O)O—, R₈—SO₂—NH—, phtalimido, succinimido, R₉C(O)—, R₁₀—(CH₂)ₙ—C(O)— or (R₁₁ₐ)(R_{11b})(R_{11c})C—C(O)O—;
(B) aryl or Het (both groups optionally substituted by one to three groups selected from C₁-C₇ alkyl, hydroxy, C₁-C₇ alkoxy, halogen, R₁₂ₐOC(O)—, (R_{12b})(R_{12c})NC(O)—, R_{12d}C(O)N(R₁₂ₑ)—, R_{12f}C(O)O—, R_{12g}OC(O)NR₁₂ₕ—, (R₁₂ⱼ)(R₁₂ₖ)NC(O)O—, aryl, benzoyl or Het⁴), R₁₃C(O)— or (R₁₄ₐ)(R_{14b})N—);
R₂, R₃ and R₄ are individually selected from linear or branched C₁-C₁₄alkyl group (which alkyl group is optionally substituted by one or more groups selected from amino, hydroxy, halogen, phenyl and R₅O—) or aryl.

In one embodiment of the invention the compound of the invention is according to formula I wherein R₁, R₂ and R₃ are individually selected from

(A) C₁-C₁₄ alkyl group, which alkyl group is optionally substituted by one or more groups selected from amino, hydroxy, halogen, R₅O—, C₃-C₁₂ cycloalkyl (which cycloalkyl is optionally substituted by one or more groups selected from C₁-C₃ alkyl, hydroxy, C₁-C₃ alkoxy, halogen, oxo, R₂₃ₐOC(O)—, (R_{23b})(R_{23c})NC(O)—, R_{23d}C(O)N(R₂₃ₑ)—, R_{23f}C(O)O—, R_{23g}OC(O)—NH—, (R₂₃ₕ)(R₂₃ⱼ)NC(O)O—), aryl or Het (both groups optionally substituted by one to three groups selected from C₁-C₇ alkyl, hydroxy, —CH₂OH, halogen, oxo, nitro, C₁-C₇ alkoxy, R₂₄ₐOC(O)—, (R_{24b})(R_{24c})NC(O)—, R_{24d}C(O)N(R₂₄ₑ)—, R_{24f}C(O)O—, R_{24g}OC(O)—NH—, (R₂₄ₕ)(R₂₄ⱼ)NC(O)O—, aryl, Het or R₂₅C(O)— (which aryl and Het³ are optionally substituted by one or two halogens, C₁-C₄ alkyl, hydroxy C₁-C₄alkyl, C₁-C₄ alkoxy, hydroxy C₁-C₄ alkoxy, nitro); R₆—O—(CH₂)ₘ—O—, R₇ₐOC(O)—, (R_{7b})(R_{7c})NC(O)—, R_{7d}C(O)N(R₇ₑ)—, R_{7f}C(O)O—, R_{7g}C(O)S—, R₇ₕOC(O)N(R₇ⱼ)—, (R₇ₖ)(R₇ₗ)NC(O)O—, R₇ₘOC(O)O—, R₈—SO₂—NH—, phtalimido, succinimido, R₉C(O)—, R₁₀-(CH₂)ₙ—C(O)— or (R₁₁ₐ)(R_{11b})(R_{11c})C—C(O)O—;
(B) aryl or Het² (both groups optionally substituted by one to three groups selected from C₁-C₇ alkyl, hydroxy, C₁-C₇ alkoxy, halogen, R₁₂ₐOC(O)—, (R_{12b})(R_{12c})NC(O)—, R_{12d}C(O)N(R₁₂ₑ)—, R_{12f}C(O)O—, R_{12g}OC(O)NR₁₂ₕ—, (R₁₂ⱼ)(R₁₂ₖ)NC(O)O—, aryl, benzoyl or Het⁴), R₁₃C(O)— or (R₁₄ₐ)(R_{14b})N—); and R₄ is selected from linear or branched C₁-C₆alkyl group.

In one embodiment of the invention the compound of the invention is according to formula I wherein R₁, R₂ and R₃ are defined as above and R₄ is methyl.

In one embodiment of the invention the compound of the invention is according to formula I wherein R₁ and R₂ are individually selected from

(A) C₁-C₁₄ alkyl group, which alkyl group is optionally substituted by one or more groups selected from amino, hydroxy, halogen, R₅O—, C₃-C₁₂ cycloalkyl (which cycloalkyl is optionally substituted by one or more groups selected from C₁-C₃ alkyl, hydroxy, C₁-C₃ alkoxy, halogen, oxo, R₂₃ₐOC(O)—, (R_{23b})(R_{23c})NC(O)—, R_{23d}C(O)N(R₂₃ₑ)—, R_{23f}C(O)O—, R_{23g}OC(O)—NH—, (R₂₃ₕ)(R₂₃ⱼ)NC(O)O—), aryl or Het (both groups optionally substituted by one to three groups selected from C₁-C₇ alkyl, hydroxy, —CH₂OH, halogen, oxo, nitro, C₁-C₇ alkoxy, R₂₄ₐOC(O)—, (R_{24b})(R_{24c})NC(O)—, R_{24d}C(O)N(R₂₄ₑ)—, R_{24f}C(O)O—, R_{24g}OC(O)—NH—, (R₂₄ₕ)(R₂₄ⱼ)NC(O)O—, aryl, Het or R₂₅C(O)— (which aryl and Het are optionally substituted by one or two halogens, C₁-C₄ alkyl, hydroxy C₁-C₄alkyl, C₁-C₄ alkoxy, hydroxy C₁-C₄ alkoxy, nitro); R₆—O—(CH₂)ₙ—O—, R₇ₐOC(O)—, (R_{7b})(R_{7c})NC(O)—, R_{7d}C(O)N(R₇ₑ)—, R_{7f}C(O)O—, R_{7g}C(O)S—, R₇ₕOC(O)N(R₇ⱼ)—, (R₇ₖ)(R₇₁)NC(O)O—, R₇ₘOC(O)O—, R₈—SO₂—NH—, phtalimido, succinimido, R₉C(O)—, R₁₀—(CH₂)ₙ—C(O)— or (R₁₁ₐ)(R_{11b})(R_{11c})C—C(O)O—;
(B) aryl or Het (both groups optionally substituted by one to three groups selected from C₁-C₇ alkyl, hydroxy, C₁-C₇ alkoxy, halogen, R₁₂ₐOC(O)—, (R_{12b})(R_{12c})NC(O)—, R_{12d}C(O)N(R₁₂ₑ)—, R_{12f}C(O)O—, R_{12g}OC(O)NR₁₂ₕ—, (R₁₂ⱼ)(R₁₂ₖ)NC(O)O—, aryl, benzoyl or Het⁴), R₁₃C(O)— or (R₁₄ₐ)(R_{14b})N—); and R₃ and R₄ are individually selected from, at each occurrence, linear or branched C₁-C₆alkyl group.

In one embodiment of the invention the compound of the invention is according to formula I wherein R₁ and R₂ are defined as above; and R₃ and R₄ are methyl.

In one embodiment of the invention the compound of the invention is according to formula I wherein R₁ is as defined above, R₂, R₃ and R₄ are individually selected from, at each occurrence, linear or branched C₁-C₆alkyl group.

In one embodiment of the invention the compound of the invention is according to formula I wherein R₁ is as defined above, R₂ and R₃ are individually selected from, at each occurrence, linear or branched C₁-C₆alkyl group; and R₄ is methyl.

In one embodiment of the invention the compound of the invention is according to formula I wherein R₁ is as defined above, R₂ is selected from linear or branched C₁-C₆alkyl group; and R₃ and R₄ are methyl.

In one embodiment of the invention the compound of the invention is according to formula I wherein R₁ is as defined above, R₂, R₃ and R₄ are methyl.

In one embodiment of the invention the compound of the invention is according to formula I wherein R₁ is as defined above, R₂, R₃ and R₄ are individually selected from C₁-C₄-alkyl groups.

In one embodiment of the invention the compound of the invention is according to formula I wherein R₁ and R₂ together may represent a cyclic structure containing 5 to 10 members, optionally substituted by on or more groups selected linear or branched C₁-C₅ alkyl group, amino, hydroxy, halogen or R₅O—; R₃ and R₄ are selected from linear or branched C₁-C₄ alkyl group.

In one embodiment of the invention the compound of the invention is according to formula I wherein R₁ is selected from linear or branched C₁-C₈ alkyl group (which alkyl group is optionally substituted by one or more groups selected from amino, hydroxy, halogen, R₅O— or aryl). R₂, R₃ and R₄ are individually selected from linear or branched C₁-C₄alkyl group (which alkyl group is optionally substituted by one or more groups selected from amino, hydroxy, halogen or R₅O—) or aryl.

In one embodiment of the invention the compound of the invention is according to formula I wherein R₁ is selected from linear or branched C₁-C₈ alkyl group (which alkyl group is optionally substituted by one or more groups selected from amino, hydroxy, halogen, R₅O— or phenyl). R₂, R₃ and R₄ are individually selected from linear or branched C₁-C₄alkyl group (which alkyl group is optionally substituted by one or more groups selected from amino, hydroxy, halogen or R₅O—) or phenyl.

In one embodiment R₁ is selected from linear or branched C₁-C₈ alkyl group, which alkyl group is optionally substituted by one or more groups selected from phenyl, amino, hydroxy, halogen or R₅O—. R₂, R₃ and R₄ are selected from linear or branched C₁-C₄ alkyl group, for example, methyl, ethyl, n-propyl or isopropyl.

In one embodiment R₁ and R₂ together may represent a cyclic structure containing 5 to 10 members, optionally substituted by on or more groups selected linear or branched C₁-C₅ alkyl group, amino, hydroxy, halogen or R₅O—. R₃ and R₄ are selected from linear or branched C₁-C₄ alkyl group, for example, methyl, ethyl, n-propyl or isopropyl.

In one embodiment R₁, R₂, R₃ and R₄ are as defined above, provided that R₁, R₂, R₃ and R₄ are not simultaneously C₁ alkyl group (methyl).

In one embodiment of the invention the compound of the invention is quaternary alkyl ammoniumsalt of S-5-methoxy-2-[[(4-methoxy-3,5-dimethyl-2-pyridinyl)-methyl]sulfinyl]-1H-benzimidazole (esomeprazole) of formula I

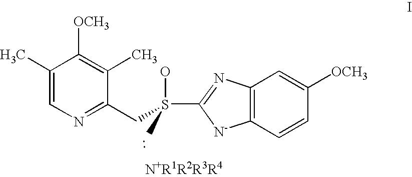

wherein
R₁, R₂, R₃ and R₄ are as defined in any place in this application, obtainable by the process described above.

Unless otherwise specified, alkyl groups and alkoxy groups as defined herein may be linear or, when there is a sufficient number (i.e. a minimum of three) of carbon atoms be branched, and/or cyclic.

As used herein, the term “C₁-C₁₄ alkyl group” is an alkyl group having 1 to 14 carbon atoms. Examples of said group includes, but is not limited to, methyl, ethyl, propyl, butyl, pentyl, hexyl, heptyl, octyl, nonyl and dekanyl and when the alkyl is branched, iso-propyl, iso-butyl, sec-butyl, tert-butyl, sec-pentyl, iso-pentyl and neo-pentyl.

The term “C₃-C₁₂ cycloalkyl” is a cyclic alkyl group having 3 to 12 carbon atoms. The cyclic group may be a mono, di or polycyclic-group, and it may optionally be substituted with 1, 2, or 3 methyl groups. Examples of said cyclic alkyl group includes, but is not limited to, cyclopropyl, cyclobutyl, cyclopentyl, cyclohexyl and adamantyl.

Unless otherwise specified, the alkyl and alkoxy groups may also be substituted by one or more fluoro atoms. Examples of said substituted alkyl or alkoxy groups are trifluoromethyl, trifluoromethoxy and trifluoroethyl.

Alkylene groups as defined herein are divalent and may be linear or, when there is a sufficient number (i.e. a minimum of three) of carbon atoms, be branched. Unless otherwise specified, alkylene groups may also be substituted by one or more halogen atoms, and especially fluoro atoms.

The term “aryl”, when used herein, includes C₆-C₁₀ aryl groups such as phenyl, naphtyl, and the like. Unless otherwise specified, the aryl group may be substituted by one or more substituents including —OH, cyano, nitro, C₁-C₇ alkoxy, C₁-C₇ alkyl, halogen for example fluoro. Examples are phenyl substituted by one, two or three halogens such as fluoro. Unless otherwise specified the term “benzoyl” also includes benzoyl groups which may be substituted by one or more halogen, for example fluoro.

Het groups (Het¹ to Het²⁷) that may be mentioned include those ring systems having a total number of atoms in the ring system or between five and twelve atoms and containing 1 to 5 heteroatoms (selected from N, O and S). Het groups may be fully saturated, wholly aromatic, partly aromatic and/or bi- or polycyclic in character. Heterocyclic groups that may be mentioned include benzodioxanyl, benzodioxepanyl, benzodioxolyl, benzofuranyl, benzimidazolyl, benzomorpholinyl, benzoxazinonyl, benzothiophenyl, chromanyl, cinnolinyl, dioxanyl, furanyl, imidazolyl, imidazo[1,2-a]pyridinyl, indolyl, isoquinolinyl, isoxazolyl, morpholinyl, oxazolyl, phthalazinyl, piperazinyl, piperidinyl, purinyl, pyranyl, pyrazinyl, pyrazolyl, pyridinyl, pyrimindinyl, pyrrolidinonyl, pyrrolidinyl, pyrrolinyl, pyrrolyl, quinazolinyl, quinolinyl, tetrahydropyranyl, tetrahydrofuranyl, thiazolyl, thienyl, thiochromanyl, triazolyl, xanthanyl and the like. Substituents on Het groups may, where appropriate, be located on any atom in the ring system including a heteroatom. The point of attachment of Het groups may be via any atom in the ring system including (where appropriate) a heteroatom, or an atom on any fused carbocyclic ring that may be present as part of the ring system. Het groups may also be in the N- or S-oxidised form. Unless otherwise specified, the Het group may be substituted by one or more substituents including —OH, cyano, nitro, C₁-C₇ alkoxy, C₁-C₇ alkyl, halogen for example fluoro.

The term “halogen”, when used herein, includes fluoro, chloro, bromo and iodo.

The phrase “R₁ and R₂ together may represent a cyclic structure containing 5-14 members” means that a mono-, bi-, tri- or polycyclic structure containing 5-14 atoms, of which optionally 1 to 5 are heteroatoms selected from N, O and S is formed. The cyclic structure may contain one or more double bond, and which cyclic structure may have one or more condensed aryl or Het. The cyclic structure may be further substituted. Examples of compounds included are pyrrolidine, piperidine, azepane, piperidone, piperazine, morpholine, tetrahydropyridine, imidazole, imidazoline, isoindoline, tetrahydroisoquinoline, carbazole, 6,7-dihydro-5H-dibenzo[c,e]azepine, 8-aza-bicyclo[3,2,1]octane, desmethyltropine, 3-oxa-9-aza-tricyclo[3.3.1.0*2,4*]nonane and desmethylscopine.

The phrase “R₁, R₂ and R₃ together may represent a cyclic structure containing 5-16 members” means that R₁, R₂ and R₃ together form a tri-, tetra- or polycyclic structure containing 5 to 16 atoms, of which optionally 1 to 5 are heteroatoms selected from N, O and S. The cyclic structure may contain one or more double bond, and which cyclic structure may have a condensed aryl or Het and which cyclic structure may optionally be further substituted by one or more groups. Examples of structures included are hexamethylenetetramine and quinuclidine.

The N(R₁)(R₂)(R₃)(R₄) X added in step (i) is defined to be salts of Cl⁻, Br⁻, I⁻, carboxylates, sulphonates, HSO₄⁻ and OH⁻. Examples of carboxylates are aliphatic carboxylic acids, for example C₁-C₆ alkyl carboxylic acid, such as acetic acid and propionic acid; of sulphonates are alkylsulphonates, for example C₁-C₆ alkyl sulphonates such as methane-, ethane- or propanesulphonic acid.

In one embodiment of the invention, the compound of the invention provided by the process above is

tetra-n-butyl ammoniumsalt of esomeprazole;
cholin salt of esomeprazole;
benzyltrimethylammonium salt of esomeprazole;
(1S)—N,N,N, trimethyl-1-phenylethylammonium salt of esomeprazole;
(1R,2S)—N,N-dimethylephedrinium salt of esomeprazole;
(1S,2R)—N,N-dimethylephedrinium salt of esomeprazole;
(1R,2S)—N-benzyl-N-methylephedrinium salt of esomeprazole;
(1S,2R)—N-benzyl-N-methylephedrinium salt of esomeprazole or
cis-2,6-dimethyl-N,N-dimethylpiperidinium salt of esomeprazole.

Due to tautomerism the chemical name (S)-5-methoxy-2-[[(4-methoxy-3,5-dimethyl-2-pyridinyl)methyl]sulfinyl]-1H-benzimidazole ammonium salt does not necessarily mean that the methoxy group of the two benzimidazole moieties is in the 5-position but may as well be in the 6-position, or there may be mixtures of the two.

The compounds of the invention may be prepared in the form of solvates, hydrates, and anhydrates.

The esomeprazole mixed in step (i) of the process of the invention is the neutral form esomeprazole, or the sodium salt or potassium salt of esomeprazole.

The process of the present invention is advantageous because of its simplicity. The process of the present of invention is defined by increased ease of handling including improved phase separation in step (iii) and an inherent drying off effect. During a phase transfer, most often a small amount of water remains in the organic phase. However, the presence of potassium carbonate in the present process reduces or even eliminates the remaining parts of aqueous solvent system in the organic phase, and thus also the need for a following drying step. It further gives products of high purity.

The compounds of the present invention are effective as gastric acid secretion inhibitors, and are thus useful as antiulcer agents. In a more general sense, they can be used for prevention and treatment of gastric-acid related conditions in mammals and especially in man, including e.g. reflux esophagitis, gastritis, duodenitis, gastric ulcer and duodenal ulcer. Furthermore, they may be used for treatment of other gastrointestinal disorders where gastric acid inhibitory effect is desirable e.g. in patients on NSAID therapy, in patients with Non Ulcer Dyspepsia, in patients with symptomatic gastro-esophageal reflux disease, and in patients with gastrinomas. They may also be used in patients in intensive care situations, in patients with acute upper gastrointestinal bleeding, pre- and postoperatively to prevent aspiration of gastric acid, to prevent and treat stress ulceration and asthma, and for improvement of sleep. Further, the compounds of the invention may be useful in the treatment of psoriasis as well as in the treatment of Helicobacter infections and related diseases. The compounds of the invention may also be used for treatment of inflammatory conditions in mammals, including man.

Any suitable route of administration may be employed for providing the patient with an effective dosage of the quartenary ammoniumsalt of esomeprazole. For example, peroral or parenteral formulations, including i.v., and the like may be employed. Dosage forms include capsules, tablets, dispersions, suspensions, solutions and the like.

It is further provided a pharmaceutical composition comprising the compounds of the present invention, as active ingredient, in association with a pharmaceutically acceptable carrier, diluent or excipient and optionally other active pharmaceutical ingredients. Compositions comprising other therapeutic ingredients are of interest in the treatment of the conditions listed above. The invention also provides the use of the compounds of the invention in the manufacture of a medicament for use in said conditions as well as a method of treating a gastric-acid related condition which method comprises administering to a subject suffering from said condition a pharmaceutically effective amount of the compounds of the invention.

The compositions of the invention include compositions suitable for peroral or parenteral administration. The compositions may be conveniently presented in unit dosage forms, and prepared by any methods known in the art of galenic pharmacy.

In the practice of the invention, the most suitable route of administration as well as the magnitude of the therapeutic dose will depend on the nature and severity of the disease to be treated. The dose, and dose frequency, may also vary according to the age, body weight and response of the individual patient. Special requirements may be needed for patients having Zollinger-Ellison syndrome, such as a need for higher doses than the average patient. Children and patients with liver diseases generally will benefit from doses that are somewhat lower than average. Thus, in some conditions it may be necessary to use doses outside the ranges stated below, for example long-term treatments may request lower dosage. Such higher and lower doses are within the scope of the present invention. Such daily doses may vary between 5 mg to 300 mg.

In general, a suitable oral dosage form of the compound of the invention may cover a dose range from 5 mg to 300 mg total daily dose, administered in one single dose or equally divided doses. A preferred dosage range is from 10 mg to 80 mg.

The compound of the invention may be combined as the active component in intimate admixture with a pharmaceutical carrier according to conventional techniques, such as the oral formulations described in WO 96/01623 and EP 0 247 983, the disclosures of which are hereby as a whole included by reference.

Combination preparations comprising the compounds of the invention and other active ingredients may also be used. Examples of such active ingredients include, but are not limited to anti-bacterial compounds, non-steroidal anti-inflammatory agents, antacid agents, alginates and prokinetic agents.

The compounds of the invention may be further processed before formulation into a suitable pharmaceutical formulation.

For the avoidance of doubt, “treatment” includes the therapeutic treatment, as well as the prophylaxis, of a condition.

### EXAMPLES

The examples below will further illustrate the preparation of the compound of the invention. These examples are not intended to limit the scope if the invention as defined hereinabove or as claimed below.

The quaternary ammoniumsalt of formula N(R₁)(R₂)(R₃)(R₄)Cl⁻ as defined above may be commercially available or otherwise synthesized according to the methods described below in Example A to Example F.

### Example A

### Preparation of (1S)—N,N,N-trimethyl-1-phenethylammonium Chloride

(1S)—N,N-dimethyl-1-phenetylamine (0.61 g, (4 mmol)) was dissolved in acetone (20 ml) and methyl iodide (2 g (14 mmol)) was added. The flask was sealed and the mixture was left over night at ambient temperature before it was diluted with diethyl ether (50 ml). The crystalline salt was filtered off and washed with diethyl ether. The quarternary ammonium iodide was dissolved in water (deionised) and the solution was filtered through an anion exchanger (50 ml Amberlite IRA-400; 20-50 mesh; Cl⁻ form) and eluted with deionized water. The eluate was concentrated to ca 20 ml at reduced pressure and freeze drying gave is 600 mg (3 mmol) of crystalline (1S)—N,N,N-trimethyl-1-phenetylammonium chloride.

¹H-NMR (400 MHz; CDCl₃): δ 7.59 (m, 1H), 7.43 (m, 3H), 5.33 (q, 2H), 3.34 (s, 9H), 1.81 (δ, 3H).

### Example B

### Preparation of (1R,2S)—N,N-dimethylephedrinium Chloride

(1R,2S)—N-methylephedrin (0.72 g, (4 mmol)) was dissolved in acetone (20 ml) and methyl iodide (2 g (14 mmol)) was added. The flask was sealed and the mixture was left over night at ambient temperature before it was diluted with diethyl ether (50 ml). The crystalline salt was filtered off and washed with diethyl ether. The quarternary ammonium iodide was dissolved in water (deionised) and the solution was filtered through an anion exchanger (50 ml Amberlite IRA-400; 20-50 mesh; Cl⁻ form) and eluted with deionized water. The eluate was concentrated to ca 20 ml at reduced pressure and freeze drying gave 685 mg (3 mmol) of crystalline (1R,2S)—N,N-dimethylephedrinium chloride.

¹H-NMR (400 MHz; CDCl₃): δ 7.27 (m, 2H), 7.22 (m, 2H), 5.42 (s, 1H), 3.46 (m, 1H), 3.19 (m, 1H), 3.16 (s, 9H), 1.08 (m, 3H).

### Example C

### Preparation of (1S,2R)—N,N-dimethylephedrinium Chloride

(1S,2R)—N-methylephedrin (0.72 g, (4 mmol)) was dissolved in acetone (20 ml) and methyl iodide (2 g (14 mmol)) was added. The flask was sealed and the mixture was left over night at ambient temperature before it was diluted with diethyl ether (50 ml). The crystalline salt was filtered off and washed with diethyl ether. The quarternary ammonium iodide was dissolved in water (deionised) and the solution was filtered through an anion exchanger (50 ml Amberlite IRA-400; 20-50 mesh; Cl⁻-form) and eluted with deionized water. The eluate was concentrated to ca 20 ml at reduced pressure and freeze drying gave 850 mg (3.7 mmol) of crystalline (1S,2R)—N,N-dimethylephedrinium chloride.

¹H-NMR (400 MHz; CDCl₃): δ 7.27 (m, 2H), 7.22 (m, 2H), 5.42 (s, 1H), 3.46 (m, 1H), 3.19 (m, 1H), 3.16 (s, 9H), 1.08 (m, 3H).

### Example D

### Preparation of (1R,2S)—N-benzyl-N-methylephedrinium Bromide

(1R,2S)—N-methylephedrin (0.5 g, (2.79 mmol)) was dissolved in dimethoxyethane (5 ml) and benzyl bromide (0.6 g (3.5 mmol)) was added. The flask was sealed and the mixture was left over night at ambient temperature before it was diluted with diethyl ether (10 ml). The crystalline salt was filtered off and washed with diethyl ether. Air drying at room temperature gave 0.74 g (2.11 mmol) of the title compound.

¹H-NMR (400 MHz; CDCl₃): δ 7.60 (d, 2H), 7.40 (bm, 5H), 7.19 (m, 2H), 7.12 (m, 1H), 5.95 (d, 1H), 5.37 (d, 1H), 5.20 (d, 1H), 4.91 (d, 1H), 3.98 (q, 1H), 3.30 (s, 3H), 3.19 (s, 3H), 1.24 (d, 3H).

### Example E

### Preparation of (1S,2R)—N-benzyl-N-methylephedrinium Bromide

(1S,2R)—N-methylephedrin (0.5 g, (2.79 mmol)) was dissolved in dimethoxyethane (5 ml) and benzyl bromide (0.6 g (3.5 mmol)) was added. The flask was sealed and the mixture was left over night at ambient temperature before it was diluted with diethyl ether (10 ml). The crystalline salt was filtered off and washed with diethyl ether. Air drying at room temperature gave 0.75 g (2.14 mmol) of the title compound.

¹H-NMR (400 MHz; CDCl₃): δ 7.60 (d, 2H), 7.40 (bm, 5H), 7.19 (m, 2H), 7.12 (m, 1H), 5.95 (d, 1H), 5.37 (d, 1H), 5.20 (d, 1H), 4.91 (d, 1H), 3.98 (q, 1H), 3.30 (s, 3H), 3.19 (s, 3H), 1.24 (d, 3H).

### Example F

### Preparation of cis-2,6-dimethyl-N,N-dimethylpiperidinium Iodide

Methyl iodide (2 g (14 mmol)) was added to a mixture of cis-2,6-dimethylpiperidine (0.46 g (4 mmol)), potassium carbonate (anhydrous) (1 g (7.3 mmol)) and water (1 ml) in dichloromethane (8 ml). The mixture was shaken carefully for 10 min whereupon the phases were separated. The organic phase was concentrated to drieness at reduced pressure and the crystalline residue was treated with acetone. Filtration and air drying gave 0.92 g (3.42 mmol) of the title compound.

¹H-NMR (400 MHz; CDCl₃): δ 4.20 (m, 2H), 3.35 (s, 3H), 2.86 (s, 3H), 1.86 (m, 6H), 1.47 (d, 6H).

### Example 1

### Preparation of tetra-n-butylammoniumsalt of Esomeprazole

Esomeprazole sodium salt (0.37 g (1 mmol)) was added to a mixture of tetra-n-butylammonium chloride (0.28 g (1 mmol)), potassium carbonate (anhydrous) (1 g (7.3 mmol) and water (1 ml). Dichloromethane (8 ml) was added and the mixture was shaken by hand (1 min). After separation, the organic phase was dried over Na₂SO₄ and filtered. The filtrate was concentrated to drieness at reduced pressure. 0.58 g (0.98 mmol) of tetra-n-butylammonium salt of esomeprazole (oil) was obtained.

¹H-NMR (400 MHz; CDCl₃): δ 8.21 (s, 1H), 7.54 (d, 1H), 7.18 (m, 1H), 6.74 (dm, 1H), 4.94 (d, 1H), 4.65 (D, 1H), 3.82 (s, 3H), 3.63 (s, 3H), 2.97 (bm, 8H), 2.19 (s, 3H), 2.18 (s, 3H), 1.29 (bm, 16H), 0.93 (bt, 12H).

### Example 2

### Preparation of Cholin Salt of Esomeprazole

Esomeprazole sodium salt (0.37 g (1 mmol)) was added to a mixture of cholin chloride (0.14 g (1 mmol)), potassium carbonate (anhydrous) (1 g (7.3 mmol) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.44 g (0.98 mmol) cholin salt of esomeprazole (amorphous foam) was obtained.

¹H-NMR (400 MHz; CDCl₃): δ 8.14 (s, 1H), 7.50 (d, 1H), 7.10 (m, 1H), 6.79 (dm, 1H), 4.83 (d, 1H), 4.57 (d, 1H), 3.84 (m, 2H), 3.82 (s, 3H), 3.67 (s, 3H), 3.27 (m, 1H), 3.10 (m, 1H), 2.93 (s, 9H), 2.20 (s, 3H), 2.19 (s, 3H).

### Example 3

### Preparation of Benzyltrimethylammonium Salt of Esomeprazole

Esomeprazole sodium salt (0.37 g (1 mmol)) was added to a mixture of benzyl trimethyl ammonium chloride (0.19 g (1 mmol)), potassium carbonate (anhydrous) (1 g (7.3 mmol) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.48 g (0.97 mmol) of benzyltrimethylammonium salt of esomeprazole (oil) was obtained.

¹H-NMR (400 MHz; CDCl₃): δ 8.12 (s, 1H), 7.52 (d, 1H), 7.38 (bm, 3H), 7.21 (bd, 2H), 7.14 (m, 1H), 6.74 (dm, 1H), 4.91 (d, 1H), 4.62 (d, 1H), 4.13 (s, 2H), 3.76 (s, 3H), 3.65 (s, 3H), 2.74 (s, 9H), 2.20 (s, 3H), 2.15 (s, 3H).

### Example 4

### Preparation of (1S)—N,N,N, trimethyl-1-phenylethylammonium Salt of Esomeprazole

Esomeprazole sodium salt (0.37 g (1 mmol)) was added to a mixture of (1S)—N,N,N, trimethyl-1-phenylethylammonium chloride (0.2 g (1 mmol)), potassium carbonate (anhydrous) (1 g (7.3 mmol)) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.50 g (0.98 mmol) of (1S)—N,N,N, trimethyl-1-phenylethylammonium salt of esomeprazole (oil) was obtained.

¹H-NMR (400 MHz; CDCl₃): δ 8.15 (s, 1H), 7.54 (d, 1H), 7.35 (bm, 5H), 7.15 (m, 1H), 6.77 (dm, 1H), 4.92 (d, 1H), 4.66 (d, 1H), 3.78 (s, 3H), 4.57 (q, 1H) 3.65 (s, 3H), 2.84 (s, 9H), 2.20 (s, 3H), 2.17 (s, 3H), 1.54 (d, 3H).

### Example 5

### Preparation of (1R,2S)—N,N-dimethylephedrinium Salt of Esomeprazole

Esomeprazole sodium salt (0.37 g (1 mmol)) was added to a mixture of (1R,2S)—N,N-dimethylephedrinium chloride (0.23 g (1 mmol)), potassium carbonate (anhydrous) (1 g (7.3 mmol)) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.505 g (0.98 mmol) of (1R, 2S)—N,N-dimethylephedrinium salt of esomeprazole (amorphous foam) was obtained.

¹H-NMR (400 MHz; CDCl₃): δ 8.06 (s, 1H), 7.38 (bm, 3H), 7.26 (bm, 2H), 7.19 (m, 1H), 6.88 (m, 1H), 6.68 (dm, 1H), 5.83 (s, 1H), 4.65 (d, 1H), 4.42 (d, 1H), 3.65 (s, 3H), 3.56 (s, 3H), 3.10 (q, 1H), 2.86 (s, 9H), 2.14 (s, 3H), 1.93 (s, 3H), 1.10 (d, 3H).

### Example 6

### Preparation of (1S,2R)—N,N-dimethylephedrinium Salt of Esomeprazole

Esomeprazole sodium salt (0.37 g (1 mmol)) was added to a mixture of (1S,2R)—N,N-dimethylephedrinium chloride (0.23 g (1 mmol)), potassium carbonate (anhydrous) (1 g (7.3 mmol)) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.53 g (0.98 mmol) of (1S, 2R)—N,N-dimethylephedrinium salt of esomeprazole (amorphous foam) was obtained.

¹H-NMR (400 MHz; CDCl₃): δ 8.09 (s, 1H), 7.35 (bm, 3H), 7.28 (bm, 2H), 7.22 (m, 1H), 6.79 (m, 1H), 6.66 (dm, 1H), 5.60 (s, 1H), 4.76 (d, 1H), 4.52 (d, 1H), 3.62 (s, 3H), 3.57 (s, 3H), 3.13 (q, 1H), 2.96 (s, 9H), 2.17 (s, 3H), 2.09 (s, 3H), 1.12 (d, 3H).

### Example 7

### Preparation of (1R,2S)—N-benzyl-N-methylephedrinium Salt of Esomeprazole

Esomeprazole sodium salt (0.185 g (0.5 mmol)) was added to a mixture of (1R,2S)—N-benzyl-N-methylephedrinium bromide (0.175 g (0.5 mmol)), potassium carbonate (anhydrous) (1 g (7.3 mmol)) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.265 g (0.43 mmol) of (1R,2S)—N-benzyl-N-methylephedrinium salt of esomeprazole (amorphous foam) was obtained.

¹H-NMR (400 MHz; CDCl₃): δ 8.00 (s, 1H), 7.49 (d, 1H), 7.44 (d, 2H), 7.39 (t, 1H), 7.27 (bm, 3H), 7.13 (d, 2H), 6.99 (d, 1H), 6.70 (dd, 1H), 6.2 (s, 1H), 4.64 (d, 1H), 4.52 (s, 2H), 4.49 (d, 1H), 3.71 (s, 3H), 3.57 (s, 3H), 3.49 (q, 1H), 2.92 (s, 3H), 2.92 (s, 3H), 2.12 (s, 3H), 1.97 (s, 3H), 1.34 (d, 3H).

### Example 8

### Preparation of (1S,2R)—N-benzyl-N-methylephedrinium Salt of Esomeprazole

Esomeprazole sodium salt (0.185 g (0.5 mmol)) was added to a mixture of (1S,2R)—N-benzyl-N-methylephedrinium bromide (0.175 g (0.5 mmol)), potassium carbonate (anhydrous) (1 g (7.3 mmol)) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.260 g (0.42 mmol) of (1S,2R)—N-benzyl-N-methylephedrinium salt of esomeprazole (amorphous foam) was obtained.

¹H-NMR (400 MHz; CDCl₃): δ 8.01 (s, 1H), 7.40 (m, 3H), 7.37 (d, 1H), 7.27 (bm, 3H), 7.18 (d, 2H), 6.86 (d, 1H), 6.66 (dd, 1H), 5.83 (s, 1H), 4.79 (d, 1H), 4.54 (d, 1H), 4.51 (d, 1H), 4.39 (d, 1H), 3.62 (s, 6H), 3.45 (q, 1H), 2.95 (s, 3H), 2.92 (s, 3H), 2.14 (s, 3H), 2.11 (s, 3H), 1.29 (d, 3H).

### Example 9

### Preparation of cis-2,6-dimethyl-N,N-dimethylpiperidinium Salt of Esomeprazole

Esomeprazole sodium salt (0.368 g (1 mmol)) was added to a mixture of cis-2,6-dimethyl-N,N-dimethylpiperidinium iodide (0.270 g (0.5 mmol)), potassium carbonate (anhydrous) (1 g (7.3 mmol)) and water (1 ml). Dichloromethane (8 ml) was added and the mixture was shaken (1 min). After separation, the organic phase was dried over Na₂SO₄ and filtered. The filtrate was concentrated to drieness at reduced pressure. 0.470 g (0.96 mmol) cis-2,6-dimethyl-N,N-dimethylpiperidinium salt of esomeprazole (amorphous foam) was obtained.

¹H-NMR (400 MHz; CDCl₃): δ 7.90 (s, 1H), 7.43 (d, 1H), 7.04 (d, 1H), 6.66 (dd, 1H), 4.61 (d, 1H), 4.44 (d, 1H), 3.68 (s, 3H), 3.62 (s, 3H), 3.41 (bm, 2H), 2.84 (s, 3H), 2.41 (s, 3H), 2.13 (s, 3H), 2.00 (s, 3H), 1.58 (m, 4H), 1.48 (m, 2H), 1.15 (dd, 6H).

1. A process for preparation of a quaternary ammonium salt of (S)-5-methoxy-2-[[(4-methoxy-3,5-dimethyl-2-pyridinyl)-methyl]sulfinyl]-1H-benzimidazole (esomeprazole), 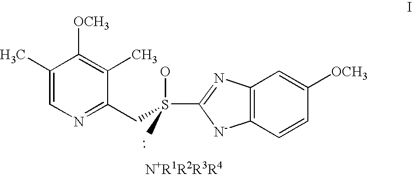
wherein:
R₁, R₂, R₃ and R₄ are individually selected from:
(A) C₁-C₁₄ alkyl group (which alkyl group is optionally substituted by one or more groups selected from amino, hydroxy, halogen, R₅O—, C₃-C₁₂ cycloalkyl (which cycloalkyl is optionally substituted by one or more groups selected from C₁-C₃ alkyl, hydroxy, C₁-C₃ alkoxy, halogen, oxo, R₂₃ₐOC(O)—, (R_{23b})(R_{23c})NC(O)—, R_{23d}C(O)N(R₂₃ₑ)—, R_{23f}C(O)O—, R_{23g}OC(O)—NH—, (R₂₃ₕ)(R₂₃ⱼ)NC(O)O—), aryl or Het¹ (both groups optionally substituted by one to three groups selected from C₁-C₇ alkyl, hydroxy, —CH₂OH, halogen, oxo, nitro, C₁-C₇ alkoxy, R₂₄OC(O)—, (R_{24b})(R_{24c})NC(O)—, R_{24d}C(O)N(R₂₄ₑ)—, R_{24f}C(O)O—, R_{24g}OC(O)—NH—, (R₂₄ₕ)(R₂₄ⱼ)NC(O)O—, aryl, Het³ or R₂₅C(O)— (which aryl and Het³ are optionally substituted by one or two halogens, C₁-C₄ alkyl, hydroxy C₁-C₄alkyl, C₁-C₄ alkoxy, hydroxy C₁-C₄ alkoxy, nitro)); R₆—O—(CH₂)ₘ—O—, R₇ₐOC(O)—, (R_{7b})(R_{7c})NC(O)—, R_{7d}C(O)N(R₇ₑ)—, R_{7f}C(O)O—, R_{7g}C(O)S—, R₇ₕOC(O)N(R₇ⱼ)—, (R₇ₖ)(R₇₁)NC(O)O—, R₇ₘOC(O)O—, R₈—SO₂—NH—, phtalimido, succinimido, R₅C(O)—, R₁₀-(CH₂)ₙ—C(O)—, (R₁₁ₐ)(R_{11b})(R_{11c})C—C(O)O—);
(B) aryl or Het² (both groups are optionally substituted by one to three groups selected from C₁-C₇ alkyl, hydroxy, C₁-C₇ alkoxy, halogen, R₁₂ₐOC(O)—, (R_{12b})(R_{12c})NC(O)—, R_{12d}C(O)N(R₁₂ₑ)—, R_{12f}C(O)O—, R_{12g}OC(O)NR₁₂ₕ—, (R₁₂ⱼ)(R₁₂ₖ)NC(O)O—, aryl, benzoyl or Het⁴), R₁₃C(O)—, (R₁₄ₐ)(R_{14b})N—);
or R₁ and R₂ together may represent a cyclic structure containing 5-14 members, optionally substituted by one or more groups selected from hydroxy, oxo, C₁-C₇ alkyl (which alkyl group is optionally substituted by one or more groups selected from hydroxy, halogen, aryl or Het⁷), R₁₅O—, R₁₆ₐOC(O)—, (R_{16b})(R_{16c})NC(O)—, R_{16d}C(O)N(R₁₆ₑ)—, R_{16f}C(O)O—, R_{16g}OC(O)NR₁₆ₕ—, (R₁₆ⱼ)(R₁₆ₖ)NC(O)O—, R₁₇C(O)—, aryl or Het⁵ (which aryl and Het⁵ are optionally substituted by one or more of C₁-C₇ alkyl, hydroxy, oxo, C₁-C₇ alkoxy, halogen, R₂₆ₐOC(O)—, (R_{26b})(R_{26c})NC(O)—, R_{26d}C(O)N(R₂₆ₑ)—, R_{26f}C(O)O—, R_{26g}OC(O)NH—, (R₂₆ₕ)(R₂₆ⱼ)NC(O)O—, phenyl or benzoyl (which phenyl or benzoyl are optionally substituted by one or two halogens, C₁-C₅ alkyl C(O)O—)), phtalimido, succinimido or (R₁₈ₐ)(R_{18b})(R_{18c})C—C(O)O—;
or R₁, R₂ and R₃ together may represent a cyclic structure containing 5-16 members, optionally substituted by one or more groups selected from hydroxy, oxo, C₁-C₇ alkyl (which alkyl group is optionally substituted by one or more groups selected from hydroxy, halogen, oxo, aryl or Het⁸), R₁₉O—, R₂OC(O)—, aryl and Het⁶ (which aryl and Het⁶ are optionally substituted by one to three groups selected from C₁-C₇ alkyl, hydroxy, C₁-C₇ alkoxy, halogen, oxo, R₂₇ₐOC(O)—, (R_{27b})(R_{27c})NC(O)—, R_{27d}C(O)N(R₂₇ₑ)—, R_{27f}C(O)O—, R_{27g}OC(O)—NH—, (R₂₇ₕ)(R₂₇ⱼ)NC(O)O—, phenyl or benzoyl), R₂₁ₐOC(O)—, (R_{21b})(R_{21c})NC(O)—, R_{21d}C(O)N(R₂₁ₑ)—, R_{21f}C(O)O—, R_{21g}OC(O)—NR₂₁ₕ—, (R₂₁ⱼ)(R₂₁ₖ)NC(O)O—, phtalimido, succinimido or (R₂₂ₐ)(R_{22b})(R_{22c}) C—C(O)O—;
R₅ is selected from C₁-C₆ alkyl, aryl, Het⁹ (which groups are optionally substituted by one or more groups selected from hydroxy, halogen, C₁-C₆ alkoxy);
R₆ is selected from aryl or Het¹⁰ (both groups optionally substituted by one or more groups selected from C₁-C₈ alkyl, hydroxy, C₁-C₇ alkoxy, halogen, R₂₈ₐOC(O)—, (R_{28b})(R_{28c})NC(O)—, R_{28d}C(O)N(R₂₈ₑ)—, R_{28f}C(O)O—, R_{28g}OC(O)—NH—, (R₂₈ₕ)(R₂₈ⱼ)NC(O)O—, aryl, benzoyl or Het¹¹);
R₇ₐ to R₇ₘ are independently selected, at each occurrence, from hydrogen, C₁-C₇ alkyl, aryl or Het¹² (which C₁-C₇ alkyl, aryl and Het¹² are optionally substituted by one or more groups selected from C₁-C₆ alkyl, hydroxy, C₁-C₃ alkoxy, halogen, R₂₉ₐOC(O)—, (R_{29b})(R_{29c})NC(O)—, R_{29d}C(O)N(R₂₉ₑ)—, R_{29f}C(O)O—, R_{29g}OC(O)—NH—, (R₂₉ₕ)(R₂₉ⱼ)NC(O)O—, aryl, benzoyl or Het¹³);
R₈ is selected from C₁-C₆ alkyl, aryl or Het¹⁴ (which groups are optionally substituted by one or more groups selected from C₁-C₆ alkyl);
R₉ is selected from linear or branched C₁-C₁₂ alkyl (optionally substituted by R₃₀OC(O)—), C₃-C₁₂ cycloalkyl (which cycloalkyl group is optionally further substituted by one or more groups selected from C₁-C₃ alkyl, hydroxy, C₁-C₃ alkoxy, halogen, R₃₁ₐOC(O)—, (R_{31b})(R_{31c})NC(O)—, R_{31d}C(O)NR₃₁ₑ—, R_{31f}C(O)O—, R_{31g}C(O)N(R₃₁ₕ)—, (R₃₁ⱼ)(R₃₁ₖ)NC(O)O—), aryl, benzoyl or Het¹⁵), aryl or Het¹⁶ (which aryl and Het¹⁶ are optionally substituted by one to three of the groups selected from C₁-C₆ alkyl, hydroxy, C₁-C₃ alkoxy, ethylenedioxy, halogen, R₃₂ₐOC(O)—, (R_{32b})(R_{32c})NC(O)—, R_{32d}C(O)NR₃₂ₑ—, R_{32f}C(O)O—, R_{32g}OC(O)NH—, (R₃₂ₕ)(R₃₂ⱼ)NC(O)O—), aryl, benzoyl or Het¹⁷);
R₁₀ is selected from aryl and Het¹⁸ (which groups are optionally substituted by one to three groups selected from C₁-C₃ alkyl, hydroxy, C₁-C₃ alkoxy, halogen, —COOH, ethylenedioxy);
R₁₁ₐ is selected from hydroxy or —CH₂OH;
R_{11b} is phenyl (optionally substituted by one to three groups selected from C₁-C₃ alkyl, hydroxy, C₁-C₃ alkoxy, halogen, R₃₃ₐOC(O)—, (R_{33b})(R_{33c})NC(O)—, R_{33d}C(O)N(R₃₃ₑ)—, R_{33f}C(O)O—, R_{33g}OC(O)—NH—, (R₃₃ₕ)(R₃₃ⱼ)NC(O)O—);
R_{11c} is selected from hydrogen, C₅-C₆ cycloalkyl, phenyl (which groups are optionally substituted by one to three groups selected from C₁-C₃alkyl, hydroxy, C₁-C₃alkoxy, halogen, R₃₄ₐOC(O)—, (R_{34b})(R_{34c})NC(O)—, R_{34d}C(O)N(R₃₄ₑ)—, R_{34f}C(O)O—, R_{34g}OC(O)NH—, (R₃₄ₕ)(R₃₄ⱼ)NC(O)O—);
R₁₂ₐ to R₁₂ₖ are independently selected, at each occurrence, from hydrogen, C₁-C₇alkyl, aryl, Het¹⁹ (which groups are optionally substituted by one or more groups selected from C₁-C₆alkyl, hydroxy, C₁-C₃alkoxy, halogen, R₃₅ₐOC(O)—, (R_{35b})(R_{35c})NC(O)—, R_{35d}C(O)N(R₃₅ₑ)—, R_{35f}C(O)O—, R_{35g}OC(O)—NH—, (R₃₅ₕ)(R₃₅ⱼ)NC(O)O—, aryl, benzoyl or Het²O);
R₁₃ is selected from hydrogen or C₁-C₆ alkyl;
R₁₄ₐ to R_{14b} are independently selected, at each occurrence, from hydrogen or C₁-C₆ alkyl;
R₁₅ is selected from C₁-C₆ alkyl, aryl or Het²¹ (which groups are optionally substituted by one or more groups selected from hydroxy, halogen or C₁-C₆ alkoxy);
R₁₆ₐ to R₁₆ₖ are independently selected from, at each occurrence, hydrogen, C₁-C₇ alkyl, aryl or Het²² (which groups are optionally substituted by one or more groups selected from C₁-C₆ alkyl, hydroxy, C₁-C₃ alkoxy, halogen, R₃₆ₐOC(O)—, (R_{36b})(R_{36c})NC(O)—, R_{36d}C(O)N(R₃₆ₑ)—, R_{36f}C(O)O—, R_{36g}OC(O)—NH—, (R₃₆ₕ)(R₃₆ⱼ)NC(O)O—, aryl, benzoyl or Het²³;
R₁₇ is selected from hydrogen or C₁-C₆ alkyl;
R₁₈ₐ is selected from hydroxy or —CH₂OH;
R_{18b} is phenyl (optionally substituted by one to three groups selected from C₁-C₃ alkyl, hydroxy, C₁-C₃ alkoxy, halogen, R₃₇ₐOC(O)—, (R_{37b})(R_{37c})NC(O)—, R_{37d}C(O)N(R₃₇ₑ)—, R_{37f}C(O)O—R_{37g}OC(O)—NH—, (R₃₇ₕ)(R₃₇ⱼ)NC(O)O—);
R_{18c} is selected from hydrogen, C₅-C₆ cycloalkyl or phenyl (which optionally is substituted by one to three groups selected from C₁-C₃ alkyl, hydroxy, C₁-C₃ alkoxy, halogen, R₃₈ₐOC(O)—, (R_{38b})(R_{38c})NC(O)—, R_{38d}C(O)N(R₃₈ₑ)—, R_{38f}C(O)O—, R_{38g}OC(O)—NH—, (R₃₈ₕ)(R₃₈ⱼ)NC(O)O—);
R₁₉ is selected from C₁-C₆ alkyl, aryl or Het²⁴ (which groups are optionally substituted by one or more groups selected from hydroxy, halogen or C₁-C₆ alkoxy);
R₂₀ is selected from hydrogen and C₁-C₆ alkyl;
R₂₁ₐ to R₂₁ₖ are independently selected, at each occurrence, from hydrogen, C₁-C₇ alkyl, aryl or Het²⁵ (which groups are optionally substituted by one or more groups selected from C₁-C₆ alkyl, hydroxy, C₁-C₃ alkoxy, halogen, R₃₉ₐOC(O)—, (R_{39b})(R_{39c})NC(O)—, R_{39d}C(O)N(R₃₉ₑ)—, R_{39f}C(O)O—, R_{39g}OC(O)—NH—, (R₃₉ₕ)(R₃₉ⱼ)NC(O)O—, aryl, benzoyl or Het²⁶);
R₂₂ₐ is selected from hydroxy or —CH₂OH;
R_{22b} is phenyl (optionally substituted by one to three groups selected from C₁-C₃ alkyl, hydroxy, C₁-C₃ alkoxy, halogen, R₄₀ₐOC(O)—, (R_{40b})(R_{40c})NC(O)—, R_{40d}C(O)N(R₄₀ₑ)—, R_{40f}C(O)O—, R_{40g}OC(O)NH—, (R₄₀ₕ)(R₄₀ⱼ)NC(O)O—);
R_{22c} is selected from hydrogen, C₅-C₆ cycloalkyl or phenyl (which optionally is substituted by one or three groups selected from C₁-C₃ alkyl, hydroxy, C₁-C₃ alkoxy, halogen, R₄₁ₐOC(O)—, (R_{41b})(R_{41c})NC(O)—, R_{41d}C(O)N(R₄₁ₑ)—, R_{41f}C(O)O—, R_{41g}OC(O)NH—, (R₄₁ₕ)(R₄₁ⱼ)NC(O)O—);
R₂₃ₐ to R₂₃ are independently selected, at each occurrence, from hydrogen or C₁-C₆alkyl;
R₂₄ₐ to R₂₄ⱼ are independently selected, at each occurrence, from hydrogen or C₁-C₆alkyl;
R₂₅ is selected from C₁-C₄alkyl, aryl or Het²⁷ (which aryl and Het²⁷ are optionally substituted by one or two halogens, C₁-C₄ alkyl, hydroxy C₁-C₄alkyl, C₁-C₄ alkoxy, hydroxy C₁-C₄ alkoxy, nitro);
R₂₆ₐ to R₂₆ⱼ are independently selected, at each occurrence, from hydrogen or C₁-C₆alkyl;
R₂₇ₐ to R₂₇ⱼ are independently selected, at each occurrence, from hydrogen or C₁-C₆alkyl;
R₂₈ₐ to R₂₈ⱼ are independently selected, at each occurrence, from hydrogen or C₁-C₆alkyl;
R₂₉ₐ to R₂₉ⱼ are independently selected, at each occurrence, from hydrogen or C₁-C₆alkyl;
R₃₀ is selected from hydrogen or C₁-C₆ alkyl;
R₃₁ₐ to R₃₁ₖ are independently selected, at each occurrence, from hydrogen or C₁-C₆alkyl;
R₃₂ₐ to R₃₂ᵢ are independently selected, at each occurrence, from hydrogen or C₁-C₆alkyl;
R₃₃ₐ to R₃₃ are independently selected, at each occurrence, from hydrogen or C₁-C₆alkyl;
R₃₄ₐ to R₃₄ⱼ are independently selected, at each occurrence, from hydrogen or C₁-C₆alkyl;
R₃₅ₐ to R₃₅ are independently selected, at each occurrence, from hydrogen or C₁-C₆alkyl;
R₃₆ₐ to R₃₆ⱼ are independently selected, at each occurrence, from hydrogen or C₁-C₆alkyl;
R₃₇ₐ to R₃₇ⱼ are independently selected, at each occurrence, from hydrogen or C₁-C₆alkyl;
R₃₈ₐ to R₃₈ⱼ are independently selected, at each occurrence, from hydrogen or C₁-C₆alkyl;
R₃₉ₐ to R₃₉ⱼ are independently selected, at each occurrence, from hydrogen or C₁-C₆alkyl;
R₄₀ₐ to R₄₀ⱼ are independently selected, at each occurrence, from hydrogen or C₁-C₆alkyl;
R₄₁ₐ to R₄₁ⱼ are independently selected, at each occurrence, from hydrogen or C₁-C₆ alkyl;
m is an integer selected from 1 to 5;
n is an integer selected from 1 to 3,
which process comprises the following steps:
(i): mixing esomeprazole and N⁺(R₁)(R₂)(R₃)(R₄)X⁻;
wherein
R₁, R₂, R₃ and R₄ are as defined above;
X⁻ is selected from Cl⁻, Br⁻, I⁻, C₁-C₆ alkyl carboxylates, C₁-C₆ alkyl sulphonates, HSO₄⁻ and OH⁻;
in an aqueous solvent system comprising more than 40% w/w potassium carbonate;
(ii): adding a water immiscible chlorinated hydrocarbon solvent;
(iii): isolating the organic phase;
(iv): recovering of the compound of formula I.

2. A process according to claim 1 wherein the aqueous solvent system in step (i) comprises more than 50% by weight potassium carbonate.

3. A process according to claim 1 wherein the aqueous solvent system in step (i) is saturated with potassium carbonate.

4. A process according to claim 1 wherein the chlorinated water immiscible solvent is selected from 1,2 dichloromethane, trichloromethane and 1,2-dichloroethane.

5. A process according to claim 1 wherein the chlorinated water immiscible solvent is dichloromethane.

6. A process according to claim 1 wherein esomeprazole in step (i) is the sodium salt or potassium salt of esomeprazole.

7. A process according to claim 1 wherein R₁ is selected from:
(A) C₁-C₁₄ alkyl group (which alkyl group is optionally substituted by one or more groups selected from amino, hydroxy, halogen, R₅₀—, C₃-C₁₂ cycloalkyl (which cycloalkyl is optionally substituted by one or more groups selected from C₁-C₃ alkyl, hydroxy, C₁-C₃ alkoxy, halogen, oxo, R₂₃ₐOC(O)—, (R_{23b})(R_{23c})NC(O)—, R_{23d}C(O)N(R₂₃ₑ)—, R_{23f}C(O)O—, R_{23g}OC(O)—NH—, (R₂₃ₕ)(R₂₃ⱼ)NC(O)O—), aryl or Het¹ (both groups optionally substituted by one to three groups selected from C₁-C₇ alkyl, hydroxy, —CH₂OH, halogen, oxo, nitro, C₁-C₇ alkoxy, R₂₄ₐOC(O)—, (R_{24b})(R_{24c})NC(O)—, R_{24d}C(O)N(R₂₄ₑ)—, R_{24f}C(O)O—, R_{24g}OC(O)—NH—, (R₂₄ₕ)(R₂₄ⱼ)NC(O)O—, aryl, Het³ or R₂₅C(O)— (which aryl, Het³ and R₂₅ are optionally substituted by one or two halogens, C₁-C₄ alkyl, hydroxy C₁-C₄alkyl, C₁-C₄ alkoxy, hydroxy C₁-C₄ alkoxy or nitro), R₆—O—(CH₂)ₘ—O—, R₇ₐOC(O)—, (R_{7b})(R_{7c})NC(O)—, R_{7d}C(O)N(R₇ₑ)—, R_{7f}C(O)O—, R_{7g}C(O)S—, R₇ₕOC(O)N(R₇ⱼ)—, (R₇ₖ)(R₇₁)NC(O)O—, R₇ₘOC(O)O—, R₈—SO₂—NH—, phtalimido, succinimido, R₉C(O)—, R₁₀—(CH₂)ₙ—C(O)—, (R₁₁ₐ)(R_{11b})(R_{11c})C—C(O)O—;
(B) aryl or Het² (both groups are optionally substituted by one to three groups selected from C₁-C₇ alkyl, hydroxy, C₁-C₇ alkoxy, halogen, R₁₂ₐOC(O)—, (R_{12b})(R_{12c})NC(O)—, R_{12d}C(O)N(R₁₂ₑ)—, R_{12f}C(O)O—, R_{12g}OC(O)NR₁₂ₕ—, (R₁₂ⱼ)(R₁₂ₖ)NC(O)O—, aryl, benzoyl or Het⁴), R₁₃C(O)—, (R₁₄ₐ)(R_{14b})N—);
R₂, R₃ and R₄ are individually selected from, at each occurrence, linear or branched C₁-C₁₄ alkyl group (which alkyl group is optionally substituted by one or more groups selected from amino, hydroxy, halogen, phenyl and R₅O—) or aryl.

8. A process according to claim 1, wherein R₄ is selected from linear or branched C₁-C₆ alkyl group.

9. A process according to claim 1, wherein R₄ is methyl.

10. A process according to claim 1, wherein R₃ and R₄ are individually selected from, at each occurrence, linear or branched C₁-C₆ alkyl group.

11. A process according to claim 1, wherein R₃ and R₄ are methyl.

12. A process according to claim 1, wherein R₂, R₃ and R₄ are individually selected from, at each occurrence, linear or branched C₁-C₆ alkyl group.

13. A process according to claim 1, wherein R₂ and R₃ are individually selected from, at each occurrence, linear or branched C₁-C₆ alkyl group; and R₄ is methyl.

14. A process according to claim 1, wherein R₂ is selected from linear or branched C₁-C₆ alkyl group; and R₃ and R₄ are methyl.

15. A process according to claim 1, wherein R₂, R₃ and R₄ are methyl.

16. A process according to claim 1, wherein R₂, R₃ and R₄ are individually selected from C₁-C₄ alkyl groups.

17. A process according to claim 1 wherein R₁ is selected from linear or branched C₁-C₈ alkyl group (which alkyl group is optionally substituted by one or more groups selected from amino, hydroxy, halogen, R₅O— or aryl); R₂, R₃ and R₄ are individually selected from linear or branched C₁-C₄alkyl group (which alkyl group is optionally substituted by one or more groups selected from amino, hydroxy, halogen or R₅O—) or aryl.

18. A process according to claim 1 wherein R₁ is selected from linear or branched C₁-C₈ alkyl group, which alkyl group is optionally substituted by one or more groups selected from amino, hydroxy, halogen, R₅O— or phenyl; R₂, R₃ and R₄ are independently, at each occurrence, selected from methyl, ethyl, n-propyl or isopropyl (which group is optionally substituted by one or more groups selected from amino, hydroxy, halogen or R₅O—) or phenyl.

19. A process according to claim 1 wherein R₁ and R₂ together may represent a cyclic structure containing 5 to 10 members, optionally substituted by one or more groups selected linear or branched C₁-C₅ alkyl group, amino, hydroxy, halogen or R₅O—; R₃ and R₄ are selected from linear or branched C₁-C₄ alkyl group.

20. Quaternary alkyl ammonium salts of S-5-methoxy-2-[[(4-methoxy-3,5-dimethyl-2-pyridinyl)-methyl]sulfinyl]-1H-benzimidazole (esomeprazole) of formula I 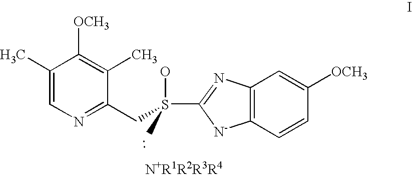
wherein
R₁, R₂, R₃ and R₄ are as defined in claim 1.

21. Quarternary ammonium salt according to claim 20, said salt being:
tetra-n-butyl ammonium salt of esomeprazole;
cholin salt of esomeprazole;
benzyltrimethylammonium salt of esomeprazole;
(1S)—N,N,N, trimethyl-1-phenylethylammonium salt of esomeprazole;
(1R,2S)—N,N-dimethylephedrinium salt of esomeprazole;
(1S,2R)—N,N-dimethylephedrinium salt of esomeprazole;
(1R,2S)—N-benzyl-N-methylephedrinium salt of esomeprazole;
(1S,2R)—N-benzyl-N-methylephedrinium salt of esomeprazole or
cis-2,6-dimethyl-N,N-dimethylpiperidinium salt of esomeprazole.

22. (canceled)

23. A pharmaceutical formulation comprising a quartenary ammonium salt of esomeprazole as defined in claim 20, in admixture with at least one pharmaceutically acceptable excipient.

24. A method of treatment which comprises administration of a therapeutically effective amount of a quartenary ammonium salt of esomeprazole as defined in claim 20 to a patient in need thereof.
